(19) United States
(12) Patent Application Publication
(10) Pub. No.: US 2026/0325723 A1
(43) Pub. Date: Oct. 22, 2026

(54) AN IMPLANTABLE OBJECT AND A METHOD OF PREPARING IT

(71) Applicant: REDDRESS LTD., Pardes Hana (IL)

(72) Inventor: Alon Kushnir, Givat Ada (IL)

(21) Appl. No.: 19/158,351

(22) PCT Filed: Aug. 31, 2023
(86) PCT No.: PCT/IL2023/050931

§ 371 Date: Aug. 20, 2025

(30) Foreign Application Priority Data
Feb. 21, 2023 (IL) 300830

(51) Int. Cl. A61L 31/08

(52) U.S. Cl. CPC A61L 31/08; A61L 2420/02

(57) ABSTRACT

An object that is intended to be implanted in a site within a subject is contacted with a mixture of whole blood and at least one coagulation initiator, prior to complete coagulation of said mixture. The mixture is then permitted to coagulate over part or all of the outside surfaces of the object which is subsequently implanted at the intended site.

### TECHNOLOGICAL FIELD

The present disclosure is in the medical field, in particular in the field of implantable objects intended to implanted in a patient by a medical procedure.

### BACKGROUND ART

References considered to be relevant as background to the presently disclosed subject matter are listed below:
U.S. Pat. No. 9,180,142
WO2023/119274
WO2023/037373
WO2022/180627
WO2022/168099

Acknowledgement of the above references herein is not to be inferred as meaning that these are in any way relevant to the patentability of the presently disclosed subject matter.

### GENERAL DESCRIPTION

The present disclosure provides an implantable object and a method of preparing it. The implantable object may be any object or device that is intended to be implanted in a medical procedure to a patient. According to this disclosure, the implantable object is coated, at least partially, with coagulated whole blood, typically autologous whole blood that is withdrawn from the subject prior to the procedure. The coating of coagulated whole blood aims to improve the reception or physical integration of the implantable object in the body of the patient, induce recovery of damaged tissue in the area of the implantation or tissue surrounding the object, prevent infection or inflammation or generally improve the medical outcome of the implant procedure. The patient may be a human or non-human animal subject.

The term “subject” is used herein to denote such a subject, whether human or non-human animal, in which in an implantable object is to be implanted.

The term “implantable object” is used herein to denote any object that is implanted in a subject within the framework of a medical procedure, that may be a surgery, a minimally invasive surgery, etc., that are in contact, after implantation, with a surrounding tissue. Some examples of implantable objects are provided in the description below. Others may include: an orthopedic device including nails, screws, ext., intended to be secured in a bone; a dental implant; an implantable neural stimulator; an implantable drug delivery device, e.g., an insulin pump; a pacemaker; etc.

Therefore, an aspect of the present disclosure provides a method for preparing an implantable object intended to be implanted in a subject. The method comprising contacting at least a portion of said implantable object with a mixture of whole blood and at least one coagulation initiator, prior to complete coagulation of said mixture. Namely, a portion of the implantable object contacts the whole blood when it is still in a flowable, non-coagulated state to allow the blood to adhere to the portion of the implantable object. Then, said mixture is permitted to coagulate with at least some of it adhering to said portion. The coagulation process may take between 1, 2, 3, 4, or 5 minutes and 6, 7. 8, 9, 10, 11, 12, 13, 14, or 15 min. until the whole blood mixture is fully coagulated and adheres to said portion. At times, the coagulation process may be controlled such to remove excess blood of said portions so as to remove access coagulating or coagulated blood off the surface of said portion, e.g., in order to leave a relatively uniform layer. Non-limiting examples by which this can be achieved include: some degree of agitation at an appropriate time before full coagulation; etching of excess coagulated blood; contacting the implantable object with the coagulating blood in a receiving space configured with contours that generally trace those of said object or at least those of said portion leaving a clearance to permit the coagulating blood to enter and forming, after coagulation, a substantially a substantially uniform layer.

It is to be noted that current disclosure encompasses any combination of the described embodiments and applicable to all aspect of this disclosure. In other words, any aspect of the present disclosure can be defined by any combination of the described embodiments.

In some embodiments of this disclosure, said whole blood is autologous blood of the subject.

In some embodiments, the method further comprises impregnating or immersing the implantable object within said blood mixture. In some embodiments the implantable object is fully immersed in the whole blood, and in other embodiments the implantable object is only partially immersed in the whole blood such that only a portion thereof contact the whole blood.

In some embodiments of the method, said implantable object comprises pores and said contacting comprises introducing said mixture into said pores.

In some embodiments of the method, the implantable object comprises a fabric portion, and said contacting comprises contacting said fabric portion with said mixture.

In some embodiments of the method, said implantable object is a surgical mesh. The surgical mesh may be any kind known in the art and may be made of organic materials, biological material, inorganic materials, or any combination thereof.

In some embodiments of the method, said implantable object is a cardiac implantable device.

In some embodiments of the method, said cardiac implantable device is a pacemaker.

In some embodiments, the method further comprises, prior to said contacting, adding one or more antibiotic agents to said mixture.

In some embodiments of the method, said contacting comprises introducing said implantable object into a receiving space of a receptacle device and filling said receiving space with said mixture. It is to be noted that the introduction of the implantable object and the filling of the mixture can be in any order, namely the order of the steps is interchangeable.

In some embodiments of the method, said receiving volume is defined by a contour of the receptacle device conforming with a contour, typically the external contour, of said implantable object. Typically, the implantable object is fitted into the receiving volume leaving a small gap between its external contour and the walls of the coagulation mold for allowing the filling of the mixture in said gap.

In some embodiments of the method, said at least one coagulation initiator is selected from any one of: Kaolin, Ca²⁺, Mg²⁺, negatively charged phospholipid (PL) and protamine sulfate, or any combination thereof.

In some embodiments, the method further comprises, prior to said contacting, withdrawing whole blood from the subject and mixing it with at least one coagulation initiator.

In some embodiments of the method, following said withdrawing, the method further comprising mixing the whole blood with one or more anti-coagulation agents.

In some embodiments of the method, said one or more anti-coagulation agents are selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.

In some embodiments, said portion of the implantable device is roughed to increase a surface roughness index thereof to improve the adherence of the whole blood to said portion.

In some embodiments, said portion of the implantable device is perforated to to improve the adherence of the whole blood to said portion.

Yet another aspect of the present disclosure provides a method for implanting an implantable object in a subject. The method comprising (i) preparing an implantable object according to any one of the above-described embodiments, or any combination thereof; and (ii) implanting said object in the subject.

Yet another aspect of the present disclosure provides a kit for preparing an implantable object prior to implantation in a subject. The kit comprising: (i) one or more blood withdrawal devices for allowing withdrawal of blood from the subject; (ii) at least one coagulation initiator for mixing with the withdrawn whole blood for obtaining a mixture of whole blood and at least one coagulation initiator; and (iii) a receptacle device comprises a receiving volume for receiving said object and permitting a contact of said object with said mixture.

In some embodiments of the kit, the receiving volume is defined by a contour of the receptacle device that conforms to a contour of said implantable object. Typically, the implantable object is fitted into such a contoured receiving volume leaving a small gap between its external contour and the walls of the coagulation mold for allowing the filling of the mixture in said gap and formation of a layer of coagulated blood over external surfaces of the implantable object, e.g., a substantially uniform laeyr.

In some embodiments, the kit further comprises one or more anti-coagulation agents. For example, such agents selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.

In some embodiments, the kit is intended for use in a method according to any one of the above-described embodiments, or any combination thereof.

Yet another aspect of the present disclosure provides an implantable object intended to be implanted in a subject, wherein said implantable object comprises at least a portion coated with coagulated whole blood.

In some embodiments of the implantable object, said whole blood is autologous blood of the subject.

In some embodiments of the implantable object, a majority of the entire external surface of the implantable object is coated with said whole blood.

In some embodiments of the implantable object, said whole blood comprises antibiotic agents.

In some embodiments, the implantable object is being prepared for implantation according to the method of any one of the above-described embodiments, or any combination thereof.

### EMBODIMENTS

Some embodiments of this disclosure will now be described in the following numbered paragraphs and referred to in subsequent paragraphs by reference to the numbers (in a format similar to that of claims). These embodiments intend to add onto (and not limit) the disclosure herein:
1. A method for preparing an implantable object intended to be implanted in a subject, comprising:
   contacting at least a portion of said implantable object with a mixture of whole blood and at least one coagulation initiator, prior to complete coagulation of said mixture;
   allowing said mixture to coagulate on said portion.
2. The method of embodiment 1, wherein said whole blood is autologous blood of the subject.
3. The method of embodiment 1 or 2, wherein said contacting comprises impregnating or immersing the implantable object within said mixture.
4. The method of any one of embodiments 1-3, wherein said implantable object comprises pores and said contacting comprises introducing said mixture into said pores.
5. The method of any one of embodiments 1-4, wherein the implantable object comprises a fabric portion, and said contacting comprises contacting the mixture with said fabric portion.
6. The method of any one of embodiments 1-5, wherein said implantable object is a surgical mesh.
7. The method of any one of embodiments 1-5, wherein said implantable object is a cardiac implantable device.
8. The method of claim 7, wherein said cardiac implantable device is a pacemaker.
9. The method of any one of embodiments 1-8, comprising, prior to said contacting, adding one or more antibiotic agents with said mixture.
10. The method of any one of embodiments 1-9, wherein said contacting comprises introducing said implantable object into a receiving space of a receptacle device and filling receiving space with said mixture.
11. The method of embodiment 10, wherein said receiving space is contoured such so as to accommodate said implantable object.
12. The method of any one of embodiments 1-11, wherein said at least one coagulation initiator is selected from any one of: Kaolin, Ca2+, Mg2+, negatively charged phospholipids (PL) and protamine sulfate, or any combination thereof.
13. The method of any one of embodiments 1-12, comprising, prior to said contacting, withdrawing whole blood from the subject and mixing it with at least one coagulation initiator.
14. The method of claim 13, wherein following said withdrawing, the method further comprising mixing the whole blood with one or more anti-coagulant.
15. The method of claim 14, wherein said one or more anti-coagulant are selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.
16. The method of any one of embodiments 1-15, wherein said mixture comprises an antimicrobial agent.
17. A method for implanting an implantable object in a subject, comprising: preparing an implantable object according to any one of claims 1-16; implanting said object in the subject.
18. A kit for preparing an implantable object for implantation in a subject, comprising:
   one or more blood withdrawal devices for withdrawal of blood from the subject;
   at least one coagulation initiator for mixing with the withdrawn whole blood to obtain a coagulating whole blood; and
   a receptacle device that comprises a receiving volume for receiving said object and permitting a contact of said object with said mixture.
19. The kit of embodiment 18, wherein said receiving volume is contoured to conform with a contour of said implantable object.
20. The kit of embodiment 18 or 19, comprising one or more anti-coagulation agents.
21. The kit of embodiment 20, wherein said more anti-coagulation agents are selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.
22. The kit of any one of embodiments 18-21, for use in a method of any one of claims 1-16.
23. An implantable object intended to be implanted in a subject, wherein said implantable object comprises at least a portion intended for coating by a coagulated whole blood.
24. The implantable object of embodiment 22, wherein said portion has a texture such so be able to adhere to coagulating or coagulated blood.
25. The implantable object of embodiment 23 or 24, wherein said at least a portion comprises a majority or the implantable object's external surface.
26. The implantable object of any one of embodiments 23-25, being prepared according to the method of any one of embodiments 1-16.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B are flow diagrams exemplifying different embodiments of the method for preparing an implantable device according to an aspect of the present disclosure.

### DETAILED DESCRIPTION

The following figures are provided to exemplify embodiments and realization of the invention of the present disclosure.

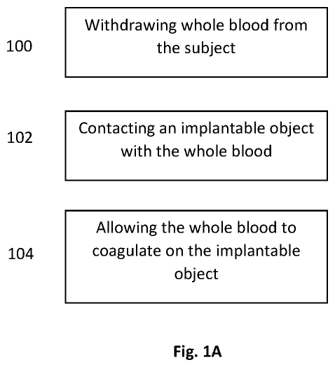

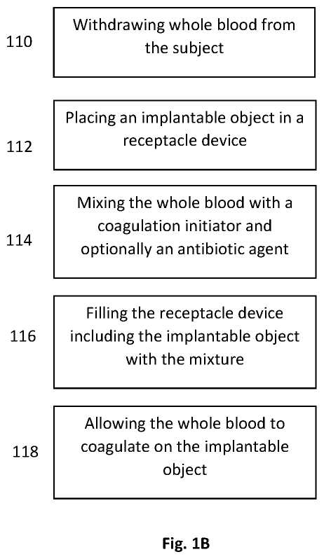

FIGS. 1A-1B are non-limiting examples of flow diagrams of different embodiments of a method for preparing an implantable device according to an aspect of the present disclosure.

FIG. 1A exemplifies a method that comprises withdrawing whole blood form the subject 100 and contacting an implantable object with the whole blood 102. Following the contact of the implantable object and the whole blood, the whole blood is allowed to be coagulated on the implantable object 104. The coagulation of the blood can be initiated, e.g., by mixing the whole blood with one or more coagulation initiators, prior to contacting the blood with the implantable object. In such a case the contact of the coagulating whole blood with the implantable object should be made at a time when the blood is still flowable and before full coagulation. Such tine window depends on various conditions including the nature of the one or more coagulation initiators and their concentration. Alternatively, the initiation of the coagulation can be made while the implantable object contacts the whole blood.

FIG. 1B exemplifies a method that comprises withdrawing whole blood form the subject 110. The method further comprises placing an implantable object in a receptacle device 112 that is shaped and sized to receive the implantable object while leaving space for filling blood such that it contacts all the portions that are desired to be coated with coagulated whole blood. The whole blood is mixed with one or more coagulation initiators 114 and optionally with one or more antimicrobial, e.g., antibiotic agents (namely, the antimicrobial agents are not mandatory) to obtain a coagulation mixture. The method further comprises filling the receptacle device with the coagulating blood mixture 116 to allow contact of the mixture with portions or the external surface of the implantable device 116 and allowing the whole blood to coagulate on the implantable object 118. It is to be noted that the method steps 110, 112, 114, 116, are interchangeable, namely the order of their appearance in the description is not binding and they can be performed in various different orders.

1. A kit for preparing an implantable object for implantation in a subject, comprising:
one or more blood withdrawal devices for withdrawal of blood from the subject;
at least one coagulation initiator for mixing with the withdrawn whole blood to obtain a coagulating whole blood; and
a receptacle device that comprises a receiving volume for receiving said object and permitting a contact of said object with said mixture.

2. The kit of claim 1, wherein said receiving volume of the receptacle device is contoured to conform with a contour of said implantable object.

3. An implantable object intended to be implanted in a subject, comprising at least a portion intended for coating by a coagulated whole blood.

4. The implantable object of claim 3, wherein said portion has a texture such so be able to adhere to coagulating or coagulated blood.

5. The implantable object of claim 3 or 4, wherein said at least a portion comprises most the implantable object's external surface.

6. A method for preparing an implantable object intended to be implanted in a subject, comprising:
contacting at least a portion of said implantable object with a mixture of whole blood and at least one coagulation initiator, prior to complete coagulation of said mixture;
allowing said mixture to coagulate on said portion.

7. The method of claim 6, wherein said whole blood is autologous blood of the subject.

8. The method of claim 6 or 7, comprising, prior to said contacting, adding one or more antibiotic agents with said mixture.

9. The method of claim 6, wherein said contacting comprises introducing said implantable object into a receiving space of a receptacle device and filling receiving space with said mixture.

10. The method of claim 6, wherein said at least one coagulation initiator is selected from any one of: Kaolin, Ca²⁺, Mg²⁺, negatively charged phospholipids (PL) and protamine sulfate, or any combination thereof.

11. The method of claim 6, comprising, prior to said contacting, withdrawing whole blood from the subject and mixing it with at least one coagulation initiator.

12. The method of claim 6, comprising introducing an antimicrobial agent to the mixture.

13. A method for implanting an implantable object in a subject, comprising:
preparing an implantable object according to claim 6;
implanting said object in the subject.
